(19) United States
(12) Patent Application Publication
(10) Pub. No.: US 2015/0276705 A1
(43) Pub. Date: Oct. 1, 2015

(54) ANALYTICAL APPARATUS SYSTEM, AND METHOD

(71) Applicant: SYSMEX CORPORATION, Kobe-shi (JP)

(72) Inventors: Naoya MAEDA, Kobe-shi (JP); Hiroyuki FUJINO, Kobe-shi (JP); Mieko ASADA, Kobe-shi (JP)

(21) Appl. No.: 14/669,119

(22) Filed: Mar. 26, 2015

(30) Foreign Application Priority Data
Mar. 28, 2014 (JP) 2014-068396

(51) Int. Cl. G01N 33/48; G06F 9/54; G06F 3/0484

(52) U.S. Cl. CPC G01N 33/48; G06F 3/04842; G06F 9/542

(57) ABSTRACT

A specimen analysis system includes a first information processing unit and a second information processing unit. Each unit includes a storage unit storing programs executing the information processing, and attribute information indicating attributes of the programs, and a processing unit that performs the information processing by executing the programs stored in the storage unit. The processing unit of a first apparatus acquires attribute information of programs stored in a second apparatus, extracts programs for processing from the first analytical apparatus, from programs stored in the second apparatus based on attribute information from the second apparatus. When a first specimen analysis even occurs, the first apparatus transmits a process request to the second apparatus, which stores a program selected from the extracted programs.

### CROSS REFERENCE TO RELATED APPLICATION

This application claims priority from prior Japanese Patent Application No. 2014-068396, filed on Mar. 28, 2014, entitled “ANALYTICAL APPARATUS SYSTEM, PROCESSING METHOD, AND COMPUTER PROGRAM”, the entire contents of which are incorporated herein by reference.

### BACKGROUND

The disclosure relates to an analytical apparatus system, which connects specimen analytical apparatuses via a network.

Japanese Patent Application Publication No. 2002-139502 discloses an analysis system that communicably connects an analytical apparatus to a server. An analysis program for analyzing measurement data obtained by a measurement of a specimen with the analytical apparatus is installed in the server.

When the analytical apparatus measures the specimen, the analytical apparatus transmits the obtained measurement data to the server. The server that receives the measurement data subjects the received measurement data to analytical processing by using the analysis program, and transmits analysis data obtained by the analytical processing to the analytical apparatus.

### SUMMARY

Functions and versions of programs installed on specimen analytical apparatuses vary among the apparatuses depending on the types, model numbers, operations inside a facility, and other factors of the specimen analytical apparatuses. It may be convenient if a certain specimen analytical apparatus can use a function of a program installed on another specimen analytical apparatus without installing the same program on itself. However, there has not been a technique for enabling this practice.

In view of the above, an embodiment of an analytical apparatus system includes: a first specimen analytical apparatus including a first measurement unit that measures a specimen, and a first information processing unit that processes an information regarding measurement of the specimen; and a second specimen analytical apparatus communicably connected to the first specimen analytical apparatus, the second specimen analytical apparatus including a second measurement unit that measures a specimen, and a second information processing unit that processes an information regarding of measurement the specimen, wherein each of the first information processing unit and the second information processing unit includes a storage unit that stores programs to execute the information processing, and attribute information indicating attributes of the programs, and a processing unit that executes the programs stored in the storage unit and processes an information regarding to measuring the specimen, and the processing unit of the first specimen analytical apparatus acquires the attribute information on the programs stored in the second specimen analytical apparatus, extracts programs, which are applicable to processing of an event occurring in the first specimen analytical apparatus, out of the programs stored in the second specimen analytical apparatus on the basis of the attribute information acquired from the second specimen analytical apparatus, and when the event occurs in the first specimen analytical apparatus, transmits a request to process the event to the second specimen analytical apparatus, which stores a program selected from the extracted programs.

### BRIEF DESCRIPTION OF DRAWINGS

FIG. 1 is a diagram illustrating an overall configuration of an analytical apparatus system according to an embodiment of the invention.

FIG. 2 is a block diagram illustrating a configuration of a specimen analytical apparatus.

FIG. 3 is a table representing contents stored in a storage unit of an information processing device.

FIGS. 4A to 4C are views illustrating an example of configurations of IPU programs held by information processing devices, in which FIG. 4A illustrates an example of an information processing device of a first apparatus, FIG. 4B illustrates an example of an information processing device of a second apparatus, and FIG. 4C illustrates an example of an information processing device of a third apparatus.

FIG. 5 is a flowchart illustrating a processing operation at the time of activating the specimen analytical apparatus in the analytical apparatus system.

FIG. 6 is a table representing an example of a module database stored in the information processing device of the second apparatus.

FIG. 7 is a flowchart illustrating procedures in step S112 of FIG. 5.

FIG. 8 is a flowchart illustrating procedures of processing in step S113 of FIG. 5.

FIG. 9 is a view illustrating an example of a function selection screen to be displayed on a display unit of the second apparatus.

FIG. 10 is a view illustrating an example of a menu screen to be displayed on the display unit of the second apparatus.

FIG. 11 is a flowchart illustrating a processing operation at the time of executing a module in the specimen analytical apparatus in the analytical apparatus system.

FIG. 12 is a view illustrating an example of an order registration acceptance screen.

FIG. 13 is a view illustrating an example of a job list stored in the information processing device of the second apparatus.

FIG. 14 is a flowchart illustrating a processing operation at the time of shutdown of the specimen analytical apparatus in the analytical apparatus system.

FIG. 15 is a view illustrating an example of a shutdown confirmation screen.

FIG. 16 is a flowchart illustrating procedures of module selection processing in a modified example.

FIG. 17 is a view illustrating an example of an input screen for accepting setting of a selection criterion for a module.

FIGS. 18A and 18B are diagrams illustrating aspects of transmission and reception of attribute information between the information processing devices.

FIG. 19 is a diagram illustrating another aspect of the transmission and reception of the attribute information between the information processing devices.

FIG. 20 is a diagram illustrating an overall configuration of an analytical apparatus system according to another embodiment.

### DETAILED DESCRIPTION

Embodiments are described below with reference to the drawings.

### 1. Configuration of Analytical Apparatus System

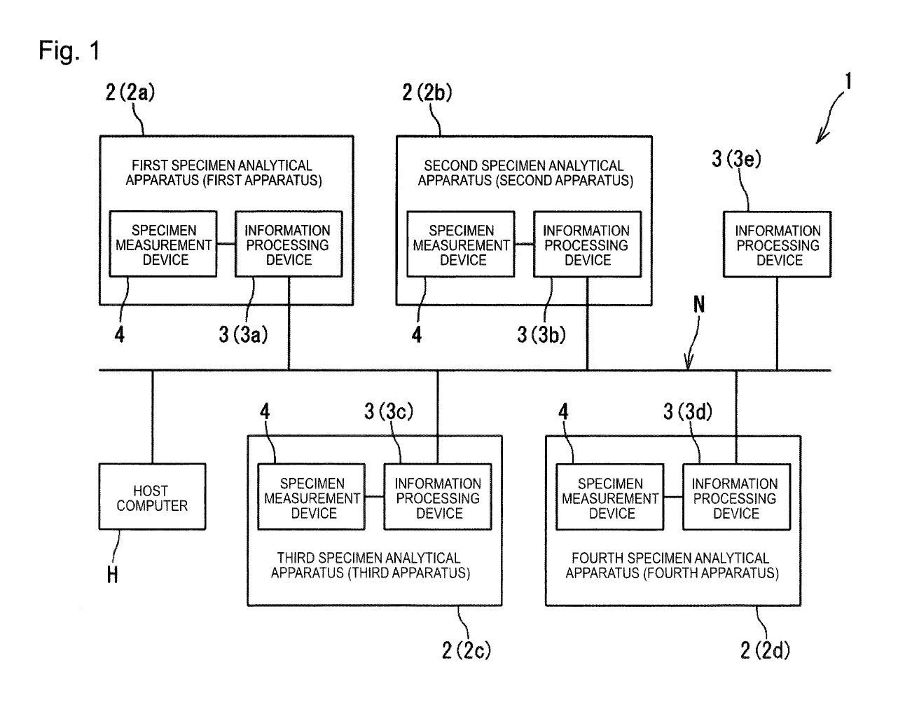

FIG. 1 is a diagram illustrating an overall configuration of an analytical apparatus system according to an embodiment of the invention.

Analytical apparatus system 1 includes specimen analytical apparatuses 2. Analytical apparatus system 1 is configured to connect specimen analytical apparatuses 2 to be communicable with one another via network N (TCP/IP network).

Specimen analytical apparatuses 2 are each an analytical apparatus configured to analyze, for example, a clinical specimen such as blood and urine collected as a specimen from a client. Each specimen analytical apparatus 2 includes information processing device 3 and specimen measurement device 4. Information processing device 3 is configured to control a specimen measurement by specimen measurement device 4, and to perform processing such as an analysis of measurement data. Note that host computer H for managing specimen measurement data and the like, and stand-alone information processing device 3 are also connected to network N of this embodiment.

Specimen analytical apparatuses 2 and information processing devices 3 may be respectively installed in different facilities or may be installed in the same facility.

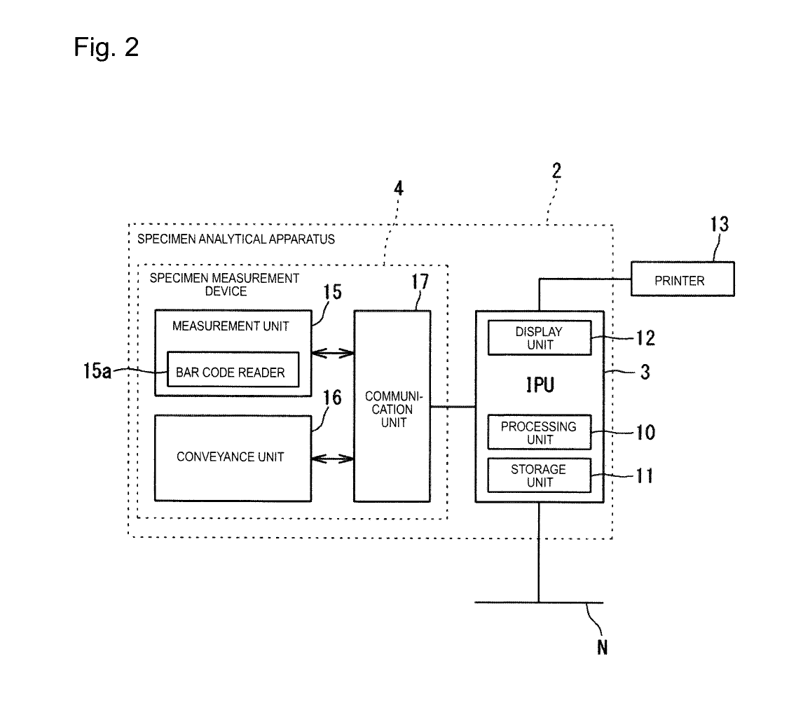

FIG. 2 is a block diagram illustrating a configuration of specimen analytical apparatus 2. Information processing device 3 included in specimen analytical apparatus 2 is equipped with processing unit 10, storage unit 11, and display unit 12, and has functions similar to a personal computer. Storage unit 11 is formed from a hard disk. Computer programs to be executed by processing unit 10 of information processing device 3 are installed on storage unit 11. Set value data necessary for execution of the processing, results of the processing, and the like are also stored in storage unit 11. The contents of storage unit 11 are described later in detail.

Processing unit 10 includes a CPU, a ROM, a RAM, and the like and has functions to read and execute the programs stored in storage unit 11. Display unit 12 is configured to perform display output of analysis results of specimens, and display of an operation screen and the like of specimen analytical apparatus 2. Display unit 12 is formed from a display device, a touch panel, or the like. Note that the analysis results can also be printed out on printer 13 connected to information processing device 3.

Specimen measurement device 4 includes measurement unit 15, conveyance unit 16, and communication unit 17. Conveyance unit 16 conveys a specimen container containing a specimen to measurement unit 15 while loading the specimen container on a rack. Measurement unit 15 suctions the specimen in the specimen container conveyed by conveyance unit 16, and performs a measurement (specimen measurement processing) concerning blood count and the like. A bar code label indicating a specimen ID for identifying each specimen is attached to the specimen container. Measurement unit 15 includes bar code reader (ID acquisition unit) 15a, which reads the bar code on the specimen container loaded on the rack. Information processing device 4 can recognize the specimen ID by reading the bar code on the specimen container by using bar code reader 15a.

Communication unit 17 has a function to perform communication between information processing device 3 and measurement unit 15 as well as conveyance unit 16. An instruction and data transmitted from information processing device 3 are provided to measurement unit 15 or conveyance unit 16 via communication unit 17, and information (such as a sensor detection result) obtained by measurement unit 15 and conveyance unit 16 is provided to information processing device 3 via communication unit 17.

Information processing device 3 of specimen analytical apparatus 2 is connected to network N, and is capable of communicating with host computer H. Host computer H is a computer which integrates and manages measurement order information to be issued to specimen analytical apparatuses 2 connected to network N, with analysis results obtained by measurements and analyses of specimens by specimen analytical apparatuses 2 in accordance with the measurement order information.

Host computer H stores and manages a client database registered by associating the measurement order information with the analysis results obtained in accordance with the measurement order information. The measurement order information is information, which indicates, for instance, an item to be measured on each specimen indicated with the specimen ID. The measurement order information includes the specimen ID, a client ID of a client from whom the specimen is collected, an item to be measured on the specimen, and the like. The measurement order information is created and registered in order registration processing to be described later.

When the specimen ID attached to the specimen is transmitted from specimen analytical apparatus 2, host computer H transmits the measurement order information corresponding to the specimen ID to information processing device 3. When information processing device 3 receives the measurement order information from host computer H, information processing device 3 stores the measurement order information in storage unit 11, and causes measurement unit 15 to perform a measurement in accordance with the measurement order information. Measurement unit 15 returns the measurement data to information processing device 3. Information processing device 3 stores the acquired measurement data in storage unit 11, and transmits an analysis result to host computer H for reporting a result in response to the measurement order information. Host computer H registers and manages the provided analysis result with the client database while associating the analysis result with the measurement order information.

### 2. Functions of Information Processing Device

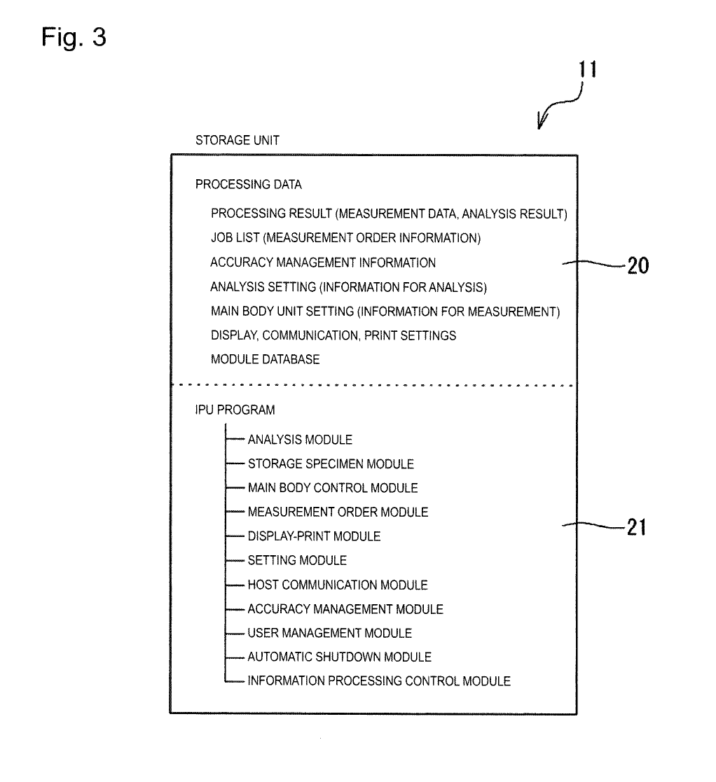

FIG. 3 is a table representing contents stored in storage unit 11 of information processing device 3. As illustrated in FIG. 3, processing data 20 and IPU program 21 are stored in storage unit 11. Processing data 20 includes: set values, which are referred to when IPU program 21 is executed; and a module database. The module database is described later in detail.

IPU program 21 includes programs each configured to perform a certain function. In this specification, a unit of a program to execute a defined function is called as a program module, or simply as a module. To be more precise, IPU program 21 includes an analysis module, a storage specimen module, a main body control module, a measurement order module, a display-print module, a setting module, a host communication module, an accuracy measurement module, a user management module, an automatic shutdown module, an information processing control module, and the like.

The analysis module is a program having a function to analyze the measurement data created by measurement unit 15 with reference to an analysis condition setting, and thereby to create an analysis result.

The storage specimen module is a program having a function to send host computer H a request to register the analysis result created by the analysis module with the client database.

The main body control module is a program having a function to cause measurement unit 15 and conveyance unit 16 to execute a measurement of a measurement item in accordance with the measurement order information. Specifically, the main body control module creates an operation schedule, which schedules an operation necessary for the operation units such as measurement unit 15 and conveyance unit 16 to measure the measurement item. To create the operation schedule, a main body unit setting included in processing data 20 is referred to. The created operation schedule is allocated to the operation units through driver circuits of measurement unit 15 and conveyance unit 16.

The measurement order module is a program having a function to accept an input of the measurement order information including the client ID and the measurement item, and a function to send host computer H a request to register the inputted measurement order information with the client database.

The display-print module is a program having functions of a data display output to display unit 12, a print data output to printer 13, and the like.

The setting module is a program having a function to accept inputs of various settings of the operation units such as measurement unit 15 and conveyance unit 16, and to store the settings in storage unit 11.

The host communication module is a program having a function to communicate with host computer H.

The accuracy management module is a program having a function to create an accuracy management chart on the basis of a result of an analysis of an accuracy management specimen.

The user management module is a program having an information management function concerning an operator who uses specimen analytical apparatus 2 and information processing device 3.

The automatic shutdown module is a program having a function to control shutdown of different device 3, which uses a module of own device 3 when shutting down own device 3.

The information processing control module has a function to comprehensively control the units of information processing device 3, and also has a function to execute processing for selectively using a module to be employed among the modules included in IPU program 21 stored in own device 3 or in different device 3 when processing an event concerning information processing occurring in own device 3.

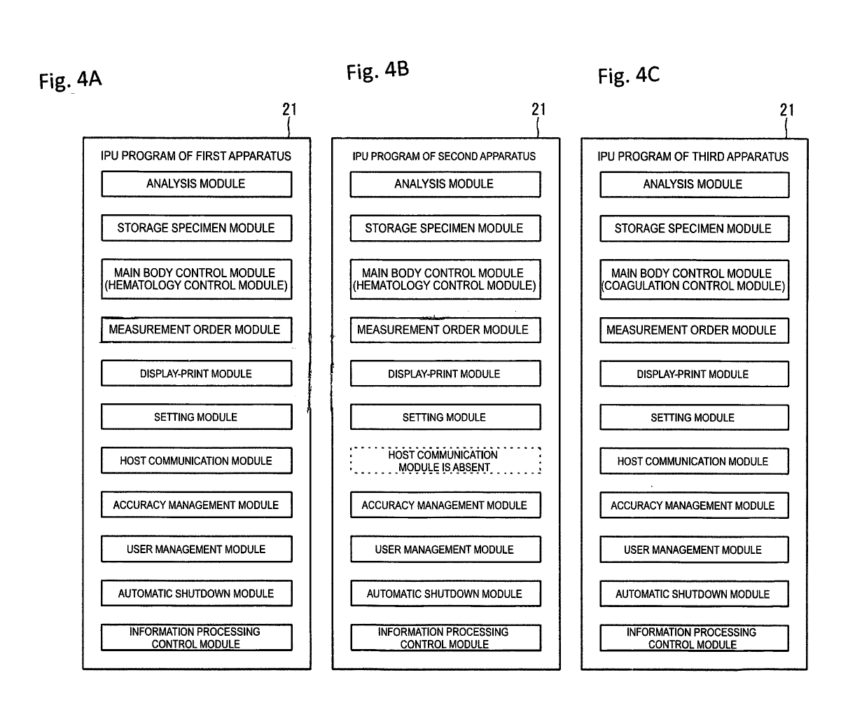

FIGS. 4A to 4C are views illustrating an example of configurations of IPU programs 21 held by information processing devices 3. FIGS. 4A, 4B, and 4C illustrate the examples of mutually different information processing devices 3. Here, each of first apparatus 2a and second apparatus 2b is assumed to be a blood cell analytical apparatus while third apparatus 2c is assumed to be a blood coagulation measurement apparatus.

As illustrated in FIGS. 4A to 4C, information processing devices 3 of apparatuses 2 may each have modules of the same function. On the other hand, a module provided to different device 3 may not be provided to own device 3. Meanwhile, even when information processing devices 3 have the modules of the same function, versions of the modules may be different from each other.

In FIGS. 4A to 4C, information processing device 3b of second apparatus 2b does not have the host communication module. However, information processing device 3b can communicate with the host computer without installing the host communication module on itself but instead by using the host communication module of any of different devices 3a and 3c. In the meantime, information processing device 3a of first apparatus 2a can execute the measurement order module installed on the own device with a different version by using the measurement order module of different device 3b. For example, if the measurement order module of information processing device 3a is of an old version while the measurement order module of information processing device 3b is of a new version, information processing device 3a can check an operation of the new version without updating the version of the measurement order module in information processing device 3a.

### 3. Concerning Processing Operations of Analytical Apparatus System

### (3.1 Processing Operation at Activation of Specimen Analytical Apparatus)

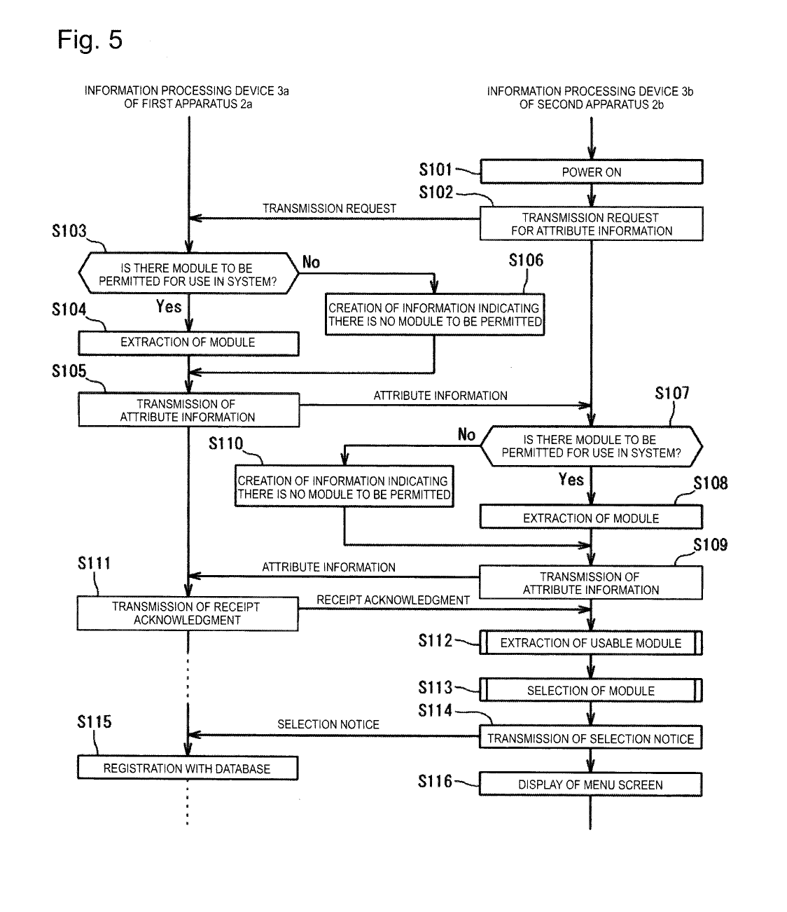

Next, a processing operation of the system of this embodiment is described. FIG. 5 is a flowchart illustrating a processing operation at the time of activating the specimen analytical apparatus in the analytical apparatus system. FIG. 5 illustrates the processing operation of information processing device 3b of second apparatus 2b at the time of activating information processing device 3b. Moreover, in order to facilitate understanding, FIG. 5 provides a description while focusing only on a relation between information processing device 3b of second apparatus 2b and information processing device 3a of first apparatus 2a, which is different device 3 in a startup state in the same system. Note that even when there are two or more different devices 3, information processing device 3b of second apparatus 2b performs substantially the same processing by involving each of different devices 3. While the processing by information processing device 3b of second apparatus 2b is mainly described herein, it is to be noted that information processing device 3a of first apparatus 2a, information processing device 3c of third apparatus 2c, and information processing device 3d of fourth apparatus 2d can also execute the same processing.

First, when information processing device 3b of second apparatus 2b is powered on, information processing device 3b is activated (step S101). By using broadcast communication, information processing device 3b of second apparatus 2b sends different devices 3 a transmission request for attribute information on modules stored in different devices 3 (step S102). The transmission request for the attribute information includes an IP address which indicates a transmission source. Here, when information processing device 3b has information concerning transmission destinations of different devices 3 connected to system 1, information processing device 3b may send the transmission request by using unicast communication.

The attribute information on the modules at least includes an apparatus type of an apparatus, which uses each module, a function type indicating the function of the module, and version information indicating the version of the module. The apparatus type includes information on a superordinate category and a subordinate category relative to the apparatus. The subordinate category is defined in accordance with the type and measurement contents of a specimen to be an analysis subject of the specimen analytical apparatus. Examples of the subordinate category include a blood count apparatus, a blood coagulation analysis apparatus, a urine analysis apparatus, an immunoanalysis apparatus, a biochemical analysis device, and the like. The superordinate category is defined as a concept that encompasses one or more subordinate categories. For example, the blood count apparatus and the urine analysis apparatus belong to the same superordinate category that is different from a superordinate category to which the blood coagulation analysis device belongs.

When information processing device 3a of first apparatus 2a receives the transmission request for the attribute information from second apparatus 2b, information processing device 3a refers to the received IP address and identifies the transmission source of the transmission request. Subsequently, information processing device 3a of first apparatus 2a determines whether or not the modules stored in own device 3a includes a module to be permitted for use by a different device (step S103). For example, if the modules stored in own device 3a include a module to be used only by own device 3a and a module, which is set in advance to restrict the use by different devices 3, then the attribute information on these modules is not transmitted.

When information processing device 3a of first apparatus 2a determines that there is the module to be permitted for use (YES in step S103), information processing device 3a extracts one or more modules to be permitted for use (step S104). Information processing device 3a of first apparatus 2a transmits the attribute information on the extracted one or more modules to the IP address indicating the transmission source of the transmission request (step S105). The attribute information to be transmitted in step S105 includes an IP address of own device 3a as information to indicate a transmission source.

On the other hand, when information processing device 3a of first apparatus 2a determines that there is no module to be permitted for use (NO in step S103), information processing device 3a creates information indicating that there is no module to be permitted for use, and transmits the information to the IP address indicating the transmission source of the transmission request (step S106). The information to be transmitted in step S106 includes the IP address of own device 3a as the information to indicate the transmission source.

When information processing device 3b of second apparatus 2b receives the information indicating the attribute information or the fact that there is no module to be permitted for use, information processing device 3b refers to the received IP address and identifies the transmission source. Thus, information processing device 3a of first apparatus 2a and information processing device 3b of second apparatus 2b can exchange their IP addresses. Accordingly, each party can specify a transmitting or receiving party in the network communication to be performed therebetween from this time on.

When information processing device 3b of second apparatus 2b receives the attribute information from information processing device 3a of first apparatus 2a, information processing device 3b determines whether or not the modules stored in own device 3b includes a module to be permitted for use by a different device (step S107).

When information processing device 3b of second apparatus 2b determines that there is the module to be permitted for use (YES in step S107), information processing device 3b extracts one or more modules to be permitted for use (step S108). Information processing device 3b of second apparatus 2b transmits the attribute information on the extracted module or modules to the IP address indicating the transmission source which has transmitted the attribute information to the own device earlier, i.e., to information processing device 3a of first apparatus 2a (step S109).

On the other hand, when information processing device 3b of second apparatus 2b determines that there is no module to be permitted for use (NO in step S107), information processing device 3b creates information indicating that there is no module to be permitted for use, and transmits the information to first apparatus 2a (step S110).

When information processing device 3a of first apparatus 2a receives the attribute information from second apparatus 2b, information processing device 3a sends information processing device 3b of second apparatus 2b a receipt acknowledgment, which notifies receipt of the attribute information from second apparatus 2b (step S111).

When information processing device 3b of second apparatus 2b receives the receipt acknowledgment from first apparatus 2a, information processing device 3b recognizes the fact that, between first apparatus 2a and second apparatus 2b, the attribute information on the modules stored in one apparatus is received and acquired by the other apparatus, and vice versa.

Information processing device 3b of second apparatus 2b registers the attribute information acquired from first apparatus 2a with the module database stored in storage unit 11.

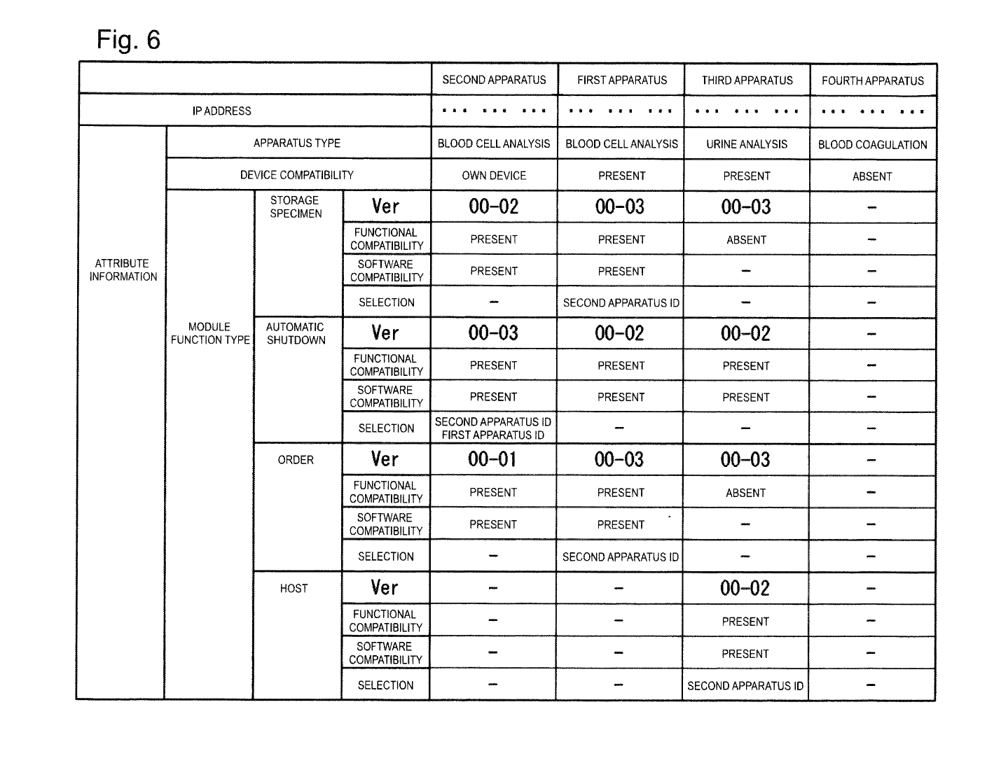

FIG. 6 is a table representing an example of the module database stored in information processing device 3b of second apparatus 2b. The attribute information transmitted from information processing devices 3 of respective apparatuses 2 connected to network N, including information processing device 3b of second apparatus 2b being the own device, is registered with the module database. The attribute information on information processing devices 3 of respective apparatuses 2 is registered in association with the IP addresses of information processing devices 3 of respective apparatuses 2, and the acquired attribute information is registered for each of devices 2.

The apparatus type, the function type, and the version included in the attribute information are registered with the module database. In the example of FIG. 6, three subordinate categories of “blood cell analysis,” “blood coagulation,” and “urine analysis” are inputted as the apparatus type. As for the function type, the information is categorized depending on the functions of the modules such as “storage specimen” which indicates the storage specimen module, “automatic shutdown” which indicates the automatic shutdown module, and “order” which indicates the measurement order module. As for the version of the modules, version numbers indicating versions of the modules are registered in columns “Ver.”

The module database is provided with columns (“functional compatibility” and “software compatibility”) for registering the presence of device compatibility between the own device and different devices, functional compatibility depending on the modules, and software compatibility.

In addition, the module database is provided with columns (“selection”) indicating the module selected by the own device in a function selection screen to be described later.

Information processing device 3b of second apparatus 2b registers the information with the module database every time the attribute information is acquired from first apparatus 2a or a determination result is obtained. Thus, information processing device 3b of second apparatus 2b stores in storage unit 11 the attribute information on the modules stored in different devices 3, and the information concerning corresponding different device 3 when a selected module is a module stored in any of different devices 3. Although the module database is stored in storage unit 11 of own device 3 in this embodiment, the module database may be stored in an external storage device other than storage unit 11 of own device 3, such as a data server connected to network N.

Back to FIG. 5, when information processing device 3b of second apparatus 2b recognizes completion of the transmission and reception of the attribute information to and from first apparatus 2a by receiving the receipt acknowledgment from first apparatus 2a, information processing device 3b extracts a module, which is usable for processing of an event occurring in the own device, on the basis of the attribute information acquired from the different devices (step S112).

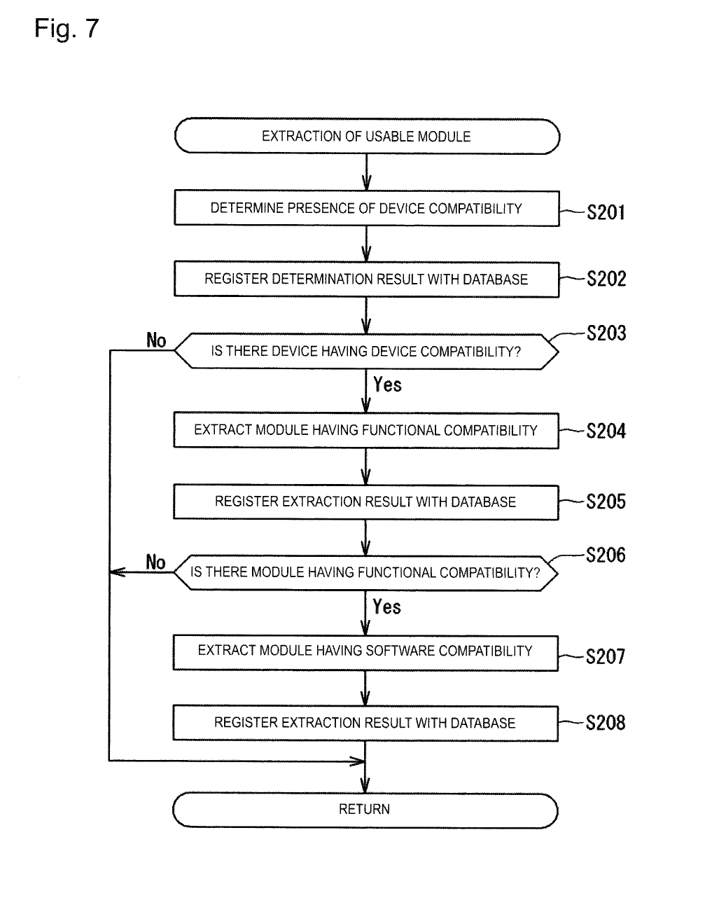

FIG. 7 is a flowchart illustrating procedures in step S112 of FIG. 5.

Information processing device 3b of second apparatus 2b determines whether or not the different device has the device compatibility with the own device on the basis of the apparatus type included in the attribute information (step S201), and registers a determination result with the module database (step S202). The determination result in step S201 is registered in the column “device compatibility” of the module database (see FIG. 6). If the determination result turns out to be “compatible,” then “present” is registered therein. If the determination result turns out to be “not compatible,” then “absent” is registered therein. Here, there may be more than one transmission sources of the attribute information. In this case, the device compatibility is determined for each of different devices 3. Here, information processing device 3b of second apparatus 2b determines the device compatibility of information processing device 3a of first apparatus 2a being different device 3 and the transmission source of the attribute information.

The determination of the device compatibility is conducted in accordance with a prescribed determination rule included in the IPU program installed on storage unit 11 by causing information processing unit 3b to execute the IPU program. In the determination rule, the apparatus type of the own device is compared with the apparatus type of the different device. If a comparison result conforms to the determination rule, then the device compatibility is determined to be present. If the comparison result does not conform to the determination rule, then the device compatibility is determined to be absent. As for the determination rule, it is possible to define a rule such as (1) that the device compatibility is determined to be present when the subordinate categories included in the apparatus type are the same, and (2) that the device compatibility is determined to be present when the superordinate categories are the same.

Subsequently, information processing device 3b of second apparatus 2b determines whether or not there is a different device determined to have the device compatibility in step S201 (step S203). If there are no different devices 3 having the device compatibility, then information processing device 3b of second apparatus 2b determines as NO in step S203, and terminates the processing.

When first apparatus 2a is determined to have the device compatibility in step S201, for example, information processing device 3b of second apparatus 2b determines as YES in step S203 and proceeds to step S204.

In step S204, information processing device 3b of second apparatus 2b extracts a module among the modules of different device 3a having the device compatibility, the module having the functional compatibility with the own device (step S204), and registers an extraction result with the module database (step S205). Information processing device 3b of second apparatus 2b extracts the module having the functional compatibility with the own device by determining whether or not the functional compatibility is present on the basis of the function type of the module included in the attribute information.

The determination of the functional compatibility is conducted in accordance with a prescribed determination rule included in the IPU program installed on storage unit 11 by causing information processing device 3b to execute the IPU program. In the determination rule, a list of the modules usable by the own device is defined in advance, and the function type is compared with this list. If the function type is included in the list, then the functional compatibility is determined to be present. If the function type is not included therein, then the functional compatibility is determined to be absent.

The determination result in step S204 is registered in the columns “functional compatibility” of the module database (see FIG. 6). Here, “present” is registered in the functional compatibility column of the module, which is determined as “having the functional compatibility,” while “absent” is registered in the functional compatibility column of the module, which is determined as “not having the functional compatibility.”

Information processing device 3b of second apparatus 2b determines whether or not there is the module of the different device having the functional compatibility (step S206). If there is not any module of the different device having the functional compatibility (NO in step S206), information processing device 3b of second apparatus 2b terminates the processing.

In step S207, information processing device 3b of second apparatus 2b extracts a module, which is compatible with the software of own device 3b, among the modules having the functional compatibility (step S207). Information processing device 3b of second apparatus 2b registers an extraction result with the module database (step S208), and terminates the processing.

Information processing device 3b of second apparatus 2b identifies the module usable with the software of own device 3b by determining whether or not the module has the compatibility with the software of own device 3b on the basis of the function type of the module and the version of the module included in the attribute information. The determination result in step S207 is registered in the columns “software compatibility” of the module database (see FIG. 6). Here, “present” is registered in the software compatibility column of the module, which is determined as “having the device compatibility,” while “absent” is registered in the functional compatibility column of the module, which is determined as “not having the device compatibility.”

The modules usable by the own device are automatically extracted on the basis of the attribute information by the processing in steps S201 to S208. Accordingly, the operator does not have to check the compatibility in terms of each module.

Back to FIG. 5, when information processing device 3b of second apparatus 2b extracts the module usable by the own device in step S112, information processing device 3b performs processing to select the module for processing the event occurring in own device 3b (step S113).

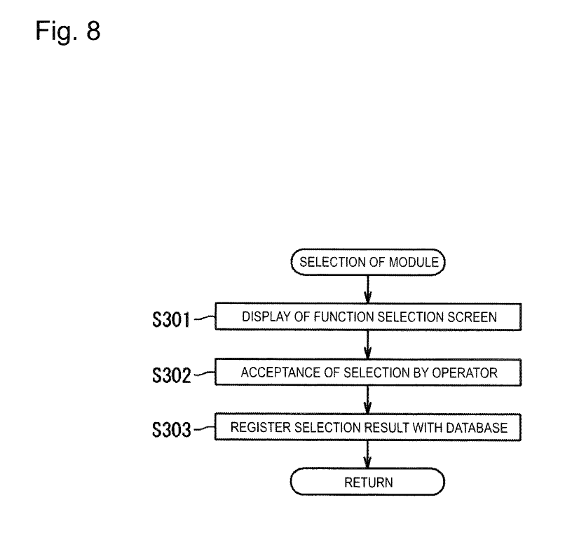

FIG. 8 is a flowchart illustrating procedures of the processing in step S113 of FIG. 5.

First, information processing device 3b of second apparatus 2b causes display unit 12 to display a function selection screen (step S301).

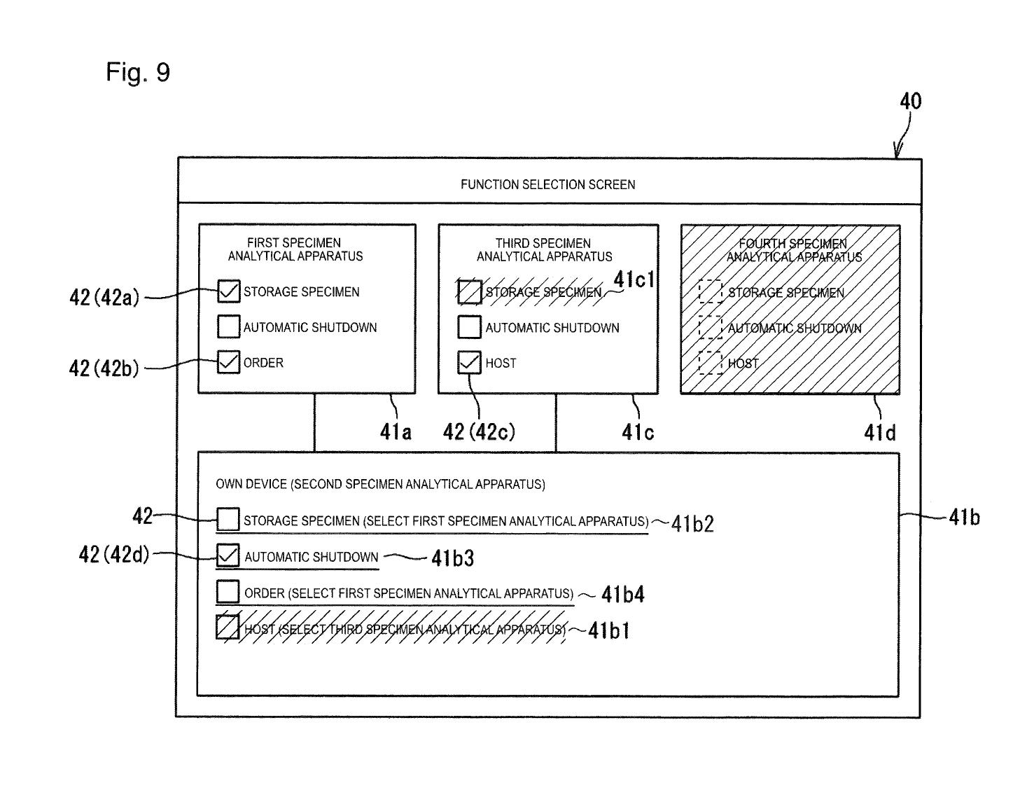

FIG. 9 is a view illustrating an example of the function selection screen to be displayed on display unit 12 of second apparatus 2b. Information processing device 3b of second apparatus 2b causes display unit 12 to display function selection screen 40 in such a way that the operator can select the modules stored in own device 3b or the modules of different devices 3 extracted in step S112.

Function selection screen 40 includes region 41b corresponding to own device 3b, and regions 41a, 41b, and 41d corresponding to different devices 3 connected to network N. Information processing device 3b of second apparatus 2b refers to the module database, and displays the names of the modules of different devices 3, which are registered with the module database, in the regions corresponding to the respective devices.

Checkbox 42 is displayed at a head portion of each of the module names in regions 41a to 41d. Information processing device 3b of second apparatus 2b can accept a result of selection by the operator by allowing the operator to perform check input in checkboxes 42 corresponding to necessary items. Note that function selection screen 40 in FIG. 9 illustrates the case of the display corresponding to the contents of the database represented in FIG. 6.

Regarding the function that cannot be selected by own device 3b, information processing device 3b of second apparatus 2b displays a portion corresponding to an unselectable item in grayout so as to restrict selection of the item by the operator. For example, the device compatibility of fourth apparatus 2d is registered as “absent” in the module database. In this case, as illustrated in FIG. 9, information processing device 3b of second apparatus 2b displays entire region 41d corresponding to fourth apparatus 2d in grayout so that the fourth apparatus 2d cannot be selected. Since the “device compatibility” of first apparatus 2a and third apparatus 2c is registered as “present,” regions 41a and 41c corresponding to these apparatuses are not displayed in grayout. Meanwhile, since the “functional compatibility” of the storage specimen module of third apparatus 2c is registered as “absent,” item display portion 41c1 corresponding to the storage specimen module in specific region 41c is displayed in grayout.

When there is a module provided to any of the different device but not provided to own device 3b, information processing device 3b of second apparatus 2b displays the name of the module in specific region 41b corresponding to own device 3b in grayout. In FIG. 9, information processing device 3b of second apparatus 2b selects the host communication module of third apparatus 2c as the module for the function not provided to own device 3b. Accordingly, as in display portion 41b1 in specific region 41b, information processing device 3b displays the module name “host” that indicates the host communication module, together with information indicating that this module is selected from (information processing device 3c) of third apparatus 2c, and displays the portion in grayout. Thus, information processing device 3c can demonstrate to the operator that the module for the function not provided to own device 3b is selected from different device 3.

On the other hand, when information processing device 3b of second apparatus 2b and different device 3 both have the modules of the same function, information processing device 3b underlines a display portion of the module name. In FIG. 9, both information processing device 3b and different device 3 have the modules of the same functions regarding the storage specimen module, the automatic shutdown module, and the measurement order module. Accordingly, information processing device 3b of second apparatus 2b underlines display portions of the module names in display portions 41b2, 41b3, and 41b4. Here, the storage specimen module and the measurement order module of first apparatus 2a are selected. Accordingly, information processing device 3b of second apparatus 2b indicates the module names together with the fact that the modules are selected from first apparatus 2a in display portions 41b2 and 41b4.

Back to FIG. 8, information processing device 3b of second apparatus 2b accepts selection of the modules by the operator through function selection screen 40 (step S302).

Information processing device 3b registers an ID of information processing device 3b of second apparatus 2b as selection information, in portions out of the “selection” columns in the module database (see FIG. 6), which are the portions corresponding to the modules selected on the function selection screen (step S303).

Information processing device 3b terminates the processing after the registration of a selection result with the module database.

Back to FIG. 5, in step S114, information processing device 3b of second apparatus 2b transmits a selection notice, which indicates that the modules stored in first apparatus 2a are selected, to first apparatus 2a that includes the selected modules (step S114). Information processing device 3b of second apparatus 2b transmits the selection notice while including the attribute information concerning the selected modules therein.

Upon receipt of the selection notice, information processing device 3a of first apparatus 2a identifies the modules selected by information processing device 3b of second apparatus 2b on the basis of the attribute information included in the selection notice. Then, information processing device 3a registers the fact that the identified modules are selected by information processing device 3b of second apparatus 2b with the module database of own device 3a (step S115).

In this case, in the “selection” column of the corresponding module at a portion of the module database where the attribute information on the modules of own device 3a is registered, information processing device 3a of first apparatus 2a registers the ID of information processing device 3b of second apparatus 2b, which is different device 3 that selects the module. For example, when information processing device 3b of second apparatus 2b is the own device, the ID of different device 3 (which is information processing device 3a of first apparatus 2a in this case) is registered in the “selection” column of “automatic shutdown” at the portion where the attribute information concerning the modules of own device 3b is registered as in the module database illustrated in FIG. 6. Thus, information processing device 3b can recognize that the automatic shutdown module stored in own device 3b is selected as the module to be used by the different device.

Information processing device 3b of second apparatus 2b causes display unit 12 to display a menu screen, which can accept an execution instruction for any of the modules (step S116).

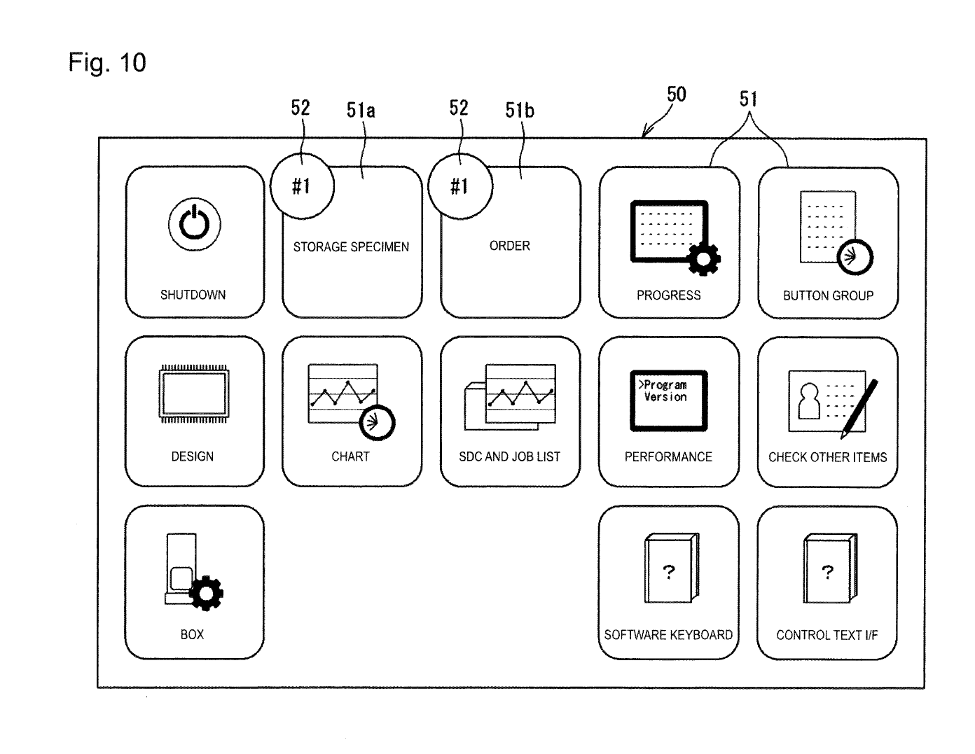

FIG. 10 is a view illustrating an example of the menu screen to be displayed on display unit 12 of second apparatus 2b. FIG. 10 illustrates a case in which information processing device 3b of second apparatus 2b performs the display in accordance with the contents of the module database represented in FIG. 6. Specifically, the modules stored in first apparatus 2a are selected as the storage specimen module and the measurement order module.

As illustrated in FIG. 10, menu screen 50 includes icons 51.

Icons 51 are provided so as to respectively correspond to the modules stored in information processing device 3b of second apparatus 2b and the modules stored in different devices 3. Accordingly, each icon 51 has a display image which enables identification as to which module icon 51 corresponds to.

Each icon 51 is displayed in such a way that icon 51 can accept an operation input by the operator. When any of the icons accepts the operation input, information processing device 3b regards the input as acceptance of an execution instruction for the module corresponding to icon 51 that accepts the operation input, and hence executes the corresponding module. For example, icon 51a displayed as “storage specimen” is the icon for executing the storage specimen module. When the operator performs the operation input of icon 51a, information processing device 3b executes the storage specimen module.

Meanwhile, concerning icons 51 corresponding to the modules stored in different devices 3, information processing device 3b of second apparatus 2b adds images thereto for identifying different devices 3 that store the modules. In the example illustrated in FIG. 10, the modules stored in information processing device 3a of first apparatus 2a are selected as the storage specimen module and the measurement order module. Accordingly, icons 51a and 51b respectively corresponding to the storage specimen module and the measurement order module are provided with images 52 for identifying the different device that stores the module corresponding to the icons.

Device display images 52 are displayed as “#1,” which mean that the storage specimen module and the measurement order module of information processing device 3a of first apparatus 2a are used as the storage specimen module and the measurement order module of information processing device 3b. This makes it possible to allow the operator to recognize as to which device the modules selected by information processing device 3b are stored in.

Back to FIG. 5, in step S116, information processing device 3b of second apparatus 2b causes display unit 12 to display menu screen 50. Thereafter, information processing device 3b maintains a stand-by state until an event concerning the specimen measurement is raised by accepting the operation input by the operator.

### (3.2 Processing Operation at the Time of Executing Module in Specimen Analytical Apparatus)

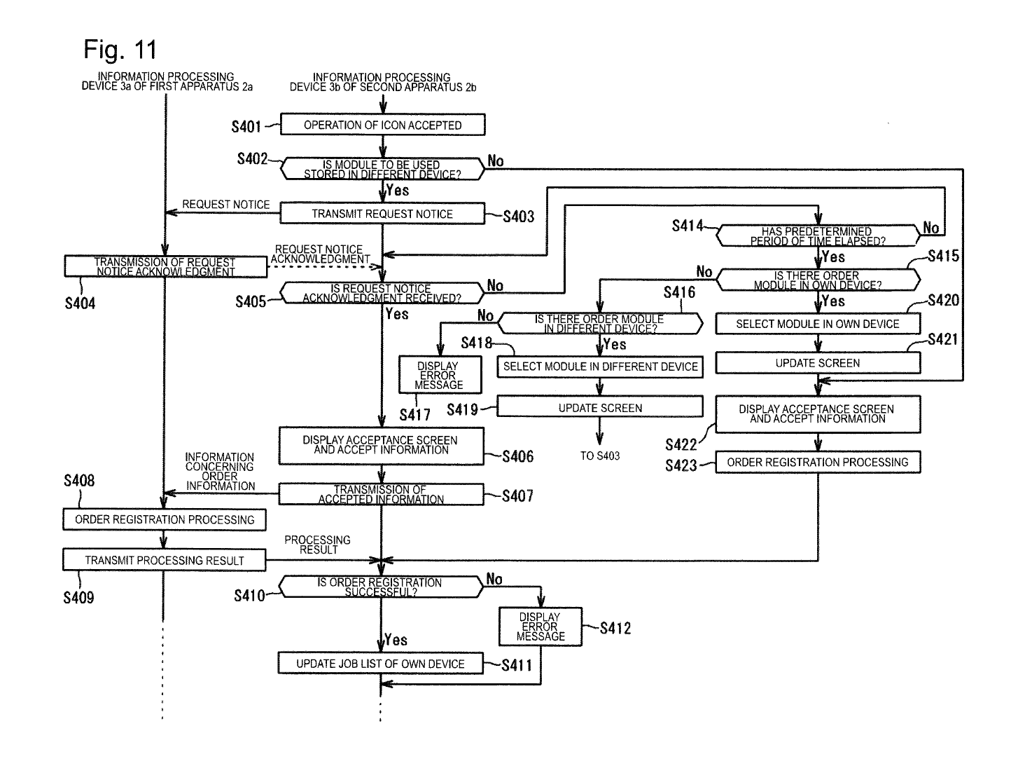

FIG. 11 is a flowchart illustrating a processing operation at the time of executing a module in the specimen analytical apparatus in the analytical apparatus system. FIG. 11 illustrates the processing operation when information processing device 3b of second apparatus 2b executes the measurement order module by accepting the operation input of icon 51b (FIG. 10) corresponding to the measurement order module included in menu screen 50 displayed on display unit 12. Moreover, in order to facilitate understanding, FIG. 11 provides a description while focusing only on a relation between second apparatus 2b and information processing device 3a of first apparatus 2a which is difference device 3 in a startup state in the same system. Note that even when there are two or more different devices 3, information processing device 3b of second apparatus 2b performs substantially the same processing by involving each of different devices 3. Furthermore, while the processing by information processing device 3b of second apparatus 2b is mainly described herein, it is to be noted that information processing device 3a of first apparatus 2a, information processing device 3c of third apparatus 2c, and information processing device 3d of fourth apparatus 2d can also execute the same processing.

First, when information processing device 3b of second apparatus 2b accepts the operation input of icon 51b by the operator (step S401), information processing device 3b refers to the module database and determines whether or not the module stored in different device 3 is selected as the module corresponding to icon 51b that accepts the operation input (step S402).

When information processing device 3b of second apparatus 2b determines that the module stored in different device 3 is selected as the module corresponding to icon 51b accepting the input, information processing device 3b transmits a request notice to different device 3 storing the selected module in order to request for the processing by the module (step S403).

In FIG. 11, assuming that different device 3 storing the relevant module is information processing device 3a of first apparatus 2a, information processing device 3b of second apparatus 2b transmits the request notice to information processing device 3a of first apparatus 2a. Information processing device 3a of first apparatus 2a, which receives the request notice sends second apparatus 2b a request notice acknowledgment, which indicates the receipt of the request notice from second apparatus 2b (step S404).

Information processing device 3b of second apparatus 2b determines whether or not the request notice acknowledgment from first apparatus 2a is received (step S405). When information processing device 3b of second apparatus 2b determines that the request notice acknowledgment is received (YES in step S405), the information processing device 3b can recognize that it is possible to establish communication connection to first apparatus 2a to which the order registration processing is to be requested. Information processing device 3b of second apparatus 2b causes display unit 12 to display order acceptance screen 60 for executing the order registration processing, and accepts the measurement order information (step S406).

Here, the operation screen (the GUI) such as order acceptance screen 60 provided for the execution of the module is created and displayed by information processing device 3b of second apparatus 2b by executing the corresponding module stored in the own device. Meanwhile, when execution of an event is instructed via the operation screen, a processing request for the event is transmitted to the different device that stores the selected module. In the example of the measurement order module, the operation screen (that is, order acceptance screen 60) is created by information processing device 3b of second apparatus 2b by executing the measurement order module stored in the own device. The event to register the measurement order with host computer H, which is a main function of the measurement order module, is processed upon an operation of OK switch 63, which is a condition to raise the event, by transmitting a processing request to information processing device 3a of first apparatus 2a together with the information on the measurement order inputted through the operation screen. Note that this is just an example and the processing is not limited only to the above-described aspect. For example, the different device may create the operation screen and the operation screen thus created may be displayed on the own device.

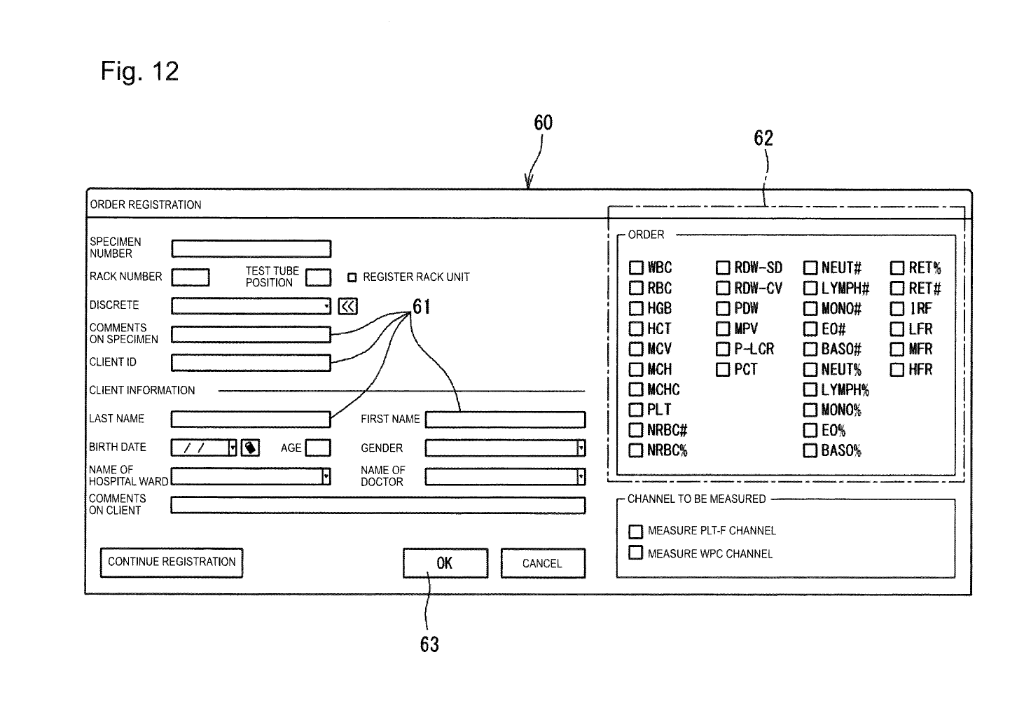

FIG. 12 is a view illustrating an example of order acceptance screen 60. Order acceptance screen 60 is provided with input boxes 61 for accepting inputs of a specimen number, information concerning a specimen with a client ID, information concerning a client, and the like. Information processing device 3b of second apparatus 2b can accept pieces of information inputted in the input boxes while associating the pieces of information with corresponding items.

Order acceptance screen 60 is provided with checkboxes 62 for selecting items to be measured with the specimen. Checkboxes 62 are respectively associated with different measurement items. The operator can perform check input by conducting the input operation with own device 3b.

Order acceptance screen 60 displays switch 63 indicating “OK,” which can accept the input operation by the operator. An operation of switch 63 by the operator raises an event to register the pieces of information inputted in the boxes on order acceptance screen 60 as the measurement order with a database of host computer H.

Back to FIG. 11, when switch 63 is operated, information processing device 3b of second apparatus 2b transmits the inputted measurement order information accepted via order acceptance screen 60 to first apparatus 2a, to which processing device 3b transmitted the request notice earlier (step S407).

When information processing device 3a of first apparatus 2a receives the measurement order information from second apparatus 2b, information processing device 3a causes the measurement order module of own device 3a to execute registration processing of the received measurement order information (step S408). Specifically, information processing device 3a of first apparatus 2a performs the processing to register the measurement order information acquired from second apparatus 2b with the database of host computer H.

As described above, information processing device 3a of first apparatus 2a processes the “execution of the order registration processing,” which is the event occurring in information processing device 3b of second apparatus 2b, on behalf of information processing device 3b of second apparatus 2b. Thus, it is possible to process the event occurring in own device 3b by using the module of different device 3.

After the completion of the order registration processing, information processing device 3a of first apparatus 2a transmits a processing result of the order registration processing to second apparatus 2b (step S409).

When information processing device 3b of second apparatus 2b receives the processing result from first apparatus 2a, information processing device 3b determines whether or not the order registration processing is successfully performed on the basis of the contents of the processing result (step S410). When the order registration processing is successfully performed (YES in step S410), information processing device 3b of second apparatus 2b updates a job list stored in storage unit 11 of own device 3b (step S411), and terminates the processing.

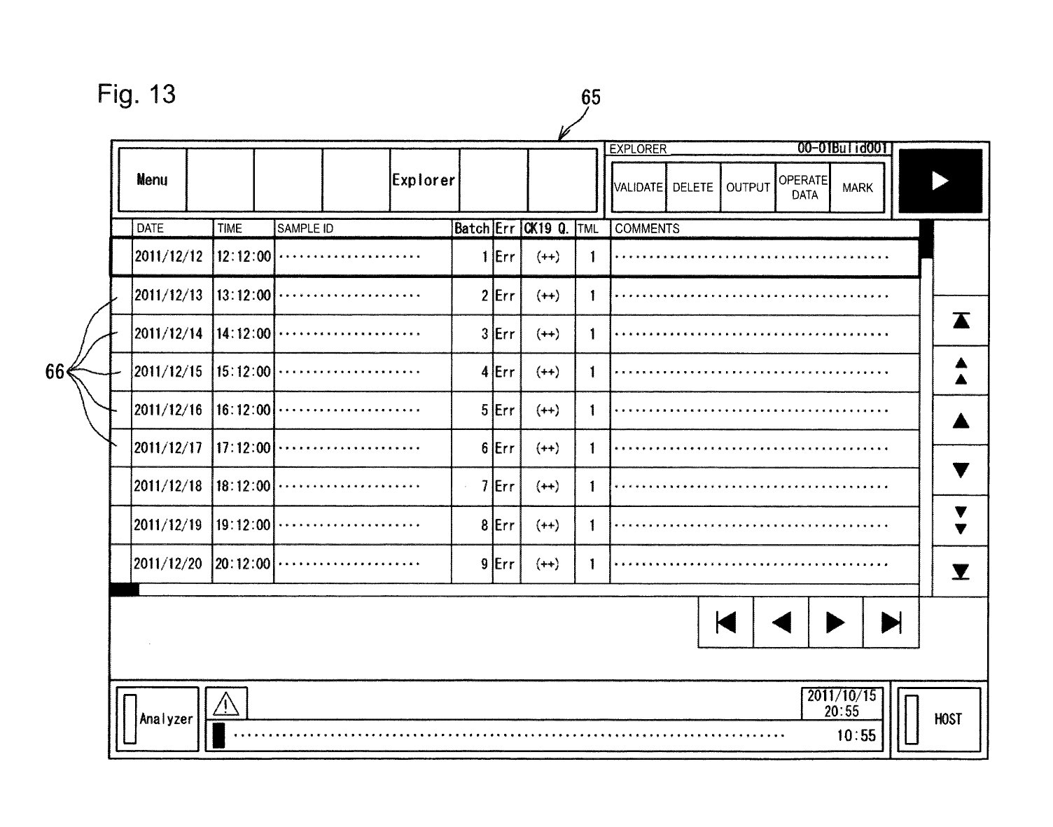

FIG. 13 is a view illustrating an example of the job list stored in information processing device 3b of second apparatus 2b. Job list 65 is a list in which the measurement order information concerning information processing device 3b of second apparatus 2b, date of the measurement, time of the measurement, the measurement data, and the like are registered.

Information processing device 3b of second apparatus 2b accesses host computer H, acquires the latest measurement order information concerning own device 3b, and updates job list 65.

Here, each device 3 has a function to include information indicating as to which device 3 performs the order registration processing into the measurement order information when performing the order registration processing. Job list 65 is provided with a column for indicating the content of the information included in the measurement order information concerning the device, which performs the order registration processing. The content of the information included in the measurement order information concerning the device, which performs the order registration processing, is indicated in the column indicated with “terminal” in FIG. 13. In FIG. 13, “1” is indicated in the column, which means that the order registration processing is performed by information processing device 3a of first apparatus 2a.

Back to FIG. 11, when the order registration processing is failed (NO in step S410), information processing device 3b of second apparatus 2b causes display unit 12 to display an error message indicating that the order registration processing is not completed due to an occurrence of an error (step S412), and terminates the processing.

In step S405 in FIG. 11, when information processing device 3b of second apparatus 2b determines that the request notice acknowledgment from first apparatus 2a is not received, information processing device 3b proceeds to step S414 and determines whether or not a predetermined period of time has elapsed since the transmission of the request notice (step S414).

When information processing device 3b of second apparatus 2b determines in step S414 that the predetermine period of time has not elapsed yet, information processing device 3b goes back to step S405 and determines again whether or not the request notice acknowledgment from first apparatus 2a is received. As described above, information processing device 3b of second apparatus 2b repeats the determination of steps S405 and S406 until the predetermined period of time elapses.

When information processing device 3b of second apparatus 2b determines that the predetermined period of time has passed without receiving the request notice acknowledgment from first apparatus 2a (step S406), information processing device 3b determines that it is not possible to establish the communication connection to first apparatus 2a, and proceeds to step S415. Here, the predetermined period of time is set to a necessary and sufficient time period (such as equal to or longer than 1 second and less than five minutes) for determining whether or not it is possible to establish a state of communication connection between devices 3a and 3b.

In step S415, information processing device 3b of second apparatus 2b determines whether or not own device 3b stores the measurement order module (step S415). When own device 3b does not store the measurement order module, information processing device 3b proceeds to step S416 and, based on the module database, determines whether or not any different device 3 other than information processing device 3a of first apparatus 2a stores the measurement order module (step S416).

If different devices 3 other than information processing device 3a of first apparatus 2a do not store the measurement order module (NO in step S416), then there are no modules supposed to execute the order registration processing under the present circumstances. Accordingly, information processing device 3b of second apparatus 2b causes display unit 12 to display an error message indicating that it is not possible to execute the order registration processing (step S417), and terminates the processing.

On the other hand, when any different device 3 other than information processing device 3a of first apparatus 2a stores the measurement order module in step S416, information processing device 3b of second apparatus 2b selects the measurement order module provided to different device 3 (step S418). Information processing device 3b of second apparatus 2b updates function selection screen 40 (FIG. 9) and menu screen 50 (FIG. 10) in accordance with the selection of the module (step S419), and returns to step S403.

Then, information processing device 3b of second apparatus 2b transmits the request notice to different device 3 which stores the measurement order module selected in step S416 (step S403). Thereafter, information processing device 3b of second apparatus 2b proceeds to step S406 when information processing device 3b receives the request notice acknowledgment (step S405), which is transmitted from different device 3 determined to include the measurement order module in step S416. The subsequent processing is the same as described above.

In step S415, if information processing device 3b of second apparatus 2b includes the measurement order module, then information processing device 3b proceeds to step S420 and selects the measurement order module of own device 3b as a module selected by own device 3b (step S420). Information processing device 3b updates function selection screen 40 (FIG. 9) and menu screen 50 (FIG. 10) in accordance with the selection of the module (step S421).

Subsequently, information processing device 3b of second apparatus 2b causes display unit 12 to display the order acceptance screen to conduct the acceptance of the measurement order information (step S422), and causes the measurement order module of own device 3b to perform the order registration processing (step S423). When the order registration processing is completed, information processing device 3b of second apparatus 2b proceeds to step S410. The subsequent processing is the same as described above.

Accordingly, when it is not possible to establish the communication connection to different device 3 having the module selected once, this embodiment is designed to select the module of the same function included in own device 3 or the module of the same function included in different device 3 other than originally selected different device. In other words, when both different device 3 and own device 3 include the modules having the same function, own device 3 is configured to select any one of the modules of the same function included in devices 3 while taking into account the state of communication with different device 3. In this case, even if the state of communication with information processing device including the previously selected module is deteriorated, own device 3 can select another module having the same function and continue the processing, which own device 3 intends to execute, as much as possible without stopping the processing.

In the above-described flowchart, if the processing result that indicates the failure of the order registration is received (NO in step S410), the error message is displayed in step S412 and the processing is terminated. However, the invention is not limited only to this sequence. If the request for processing the event is issued to the different device and the different device fails the processing, the module may be automatically reselected from the modules of the same function stored in the own device as well as other different devices. For example, in the case of NO in step S410, the processing may go to step S415 instead.

In the above-described flowchart, the measurement order registration processing is explained as an example. It is to be noted, however, that regarding other functions included in IPU program 21, the modules of different devices can also be used on the basis of a similar algorithm.

For instance, if the analysis module of the different device is selected, then receipt of the measurement data from specimen measurement device 4 corresponds to an occurrence of an event. In this case, the event is processed by transmitting a request notice to the different device storing the selected module, while requesting for an analysis of the measurement data and creation of an analysis result together with the measurement data. The different device creates the analysis result and transmits the analysis result to the transmission source.

Meanwhile, if the host communication module of the different device is selected, for example, then receipt of the specimen ID from specimen measurement device 4 corresponds to an occurrence of an event. In this case, the event is processed by transmitting a request notice to the different device storing the selected module, while requesting for an inquiry with host computer H about the measurement order on the basis of the specimen ID together with the specimen ID. The different device communicates with host computer H, inquires about the measurement order, and transmits an inquiry result to the transmission source.

### (3.3 Processing Operation at the Time of Shutdown of Specimen Analytical Apparatus)

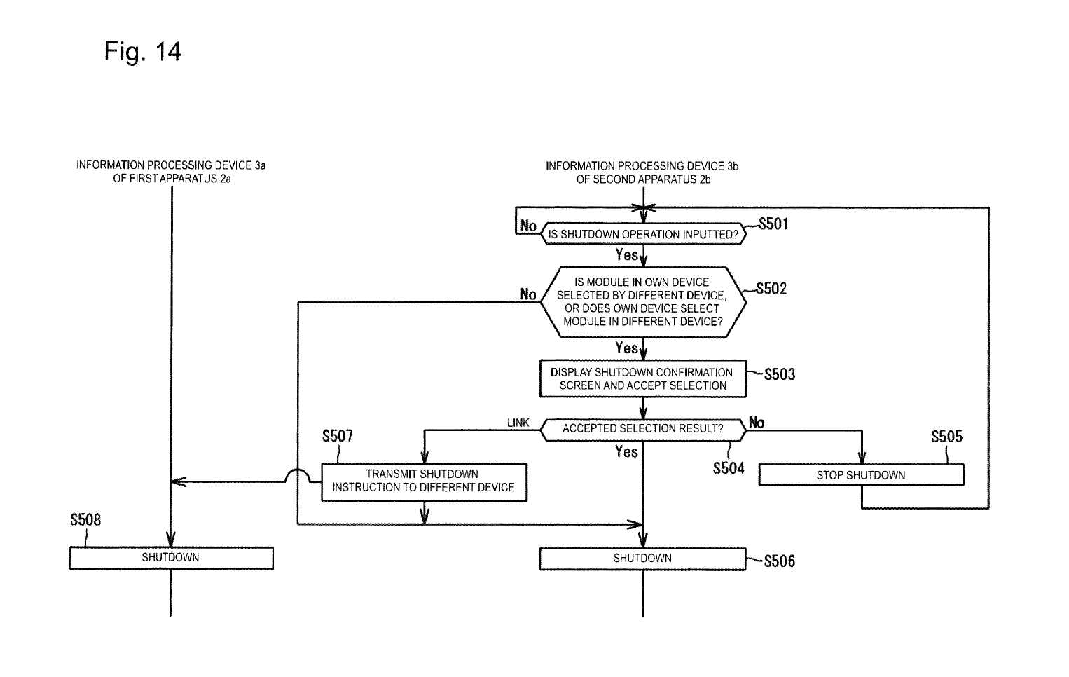

FIG. 14 is a flowchart illustrating a processing operation at the time of shutdown of the specimen analytical apparatus in the analytical apparatus system. FIG. 14 illustrates the processing operation when information processing device 3b of second apparatus 2b shuts down information processing device 3b of second apparatus 2b by accepting an operation input of shutdown. Moreover, in order to facilitate understanding, FIG. 14 provides a description while focusing only on a relation between second apparatus 2b and information processing device 3a of first apparatus 2a which is difference device 3 in a startup state in the same system. Note that even when there are two or more different devices 3, information processing device 3b of second apparatus 2b performs substantially the same processing by involving each of different devices 3. Furthermore, while the processing by information processing device 3b of second apparatus 2b is mainly described herein, it is to be noted that information processing device 3a of first apparatus 2a, information processing device 3c of third apparatus 2c, and information processing device 3d of fourth apparatus 2d can also execute the same processing.

Information processing device 3b of second apparatus 2b determines whether or not the operation input of shutdown by the operator is present (step S501). When information processing device 3b determines that the operation input of shutdown is present (YES in step S501), information processing device 3b refers to the module database and determines whether or not a module stored in own device 3b is selected by any of different devices 3, or whether or not own device 3b selects a module stored in any of different devices (step S502). As described previously, the information indicating whether or not a module stored in own device 3b is selected by any of different devices 3, and information indicating which modules are selected by own device is registered with the module database (FIG. 6). Accordingly, information processing device 3b performs the determination in step S502 by referring to the above information.

If no modules in own device 3b are selected by any of different devices 3 and if own device 3b selects no modules in different devices 3 (NO in step S502), then information processing device 3b proceeds to step S506 and executes the shutdown of own device 3b (step S506).

On the other hand, when information processing device 3b of second apparatus 2b determines that a module stored in own device 3b is selected by any of different devices 3 (YES in step S502, information processing device 3b causes display unit 12 to display a shutdown confirmation screen (step S503).

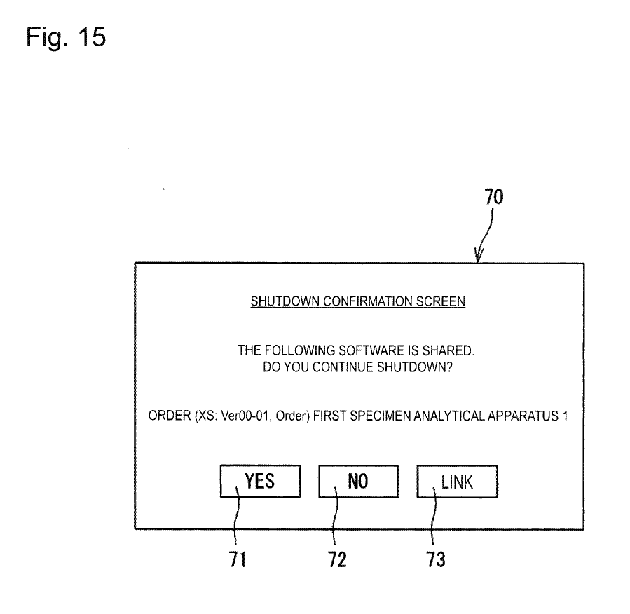

FIG. 15 is a view illustrating an example of the shutdown confirmation screen. Shutdown confirmation screen 70 displays the name of different device 3 (“first specimen analytical apparatus” in the illustrated example), which selects the module of own device 3b, and information that enables identification of the selected module (“order (XS: Ver00-01, Order)” in the illustrated example).

As described above, by displaying shutdown confirmation screen 70, information processing device 3b can allow the operator to recognize the fact that the module in own device 3b is selected by different device 3. This makes it possible to avoid a situation where a failure occurs in the processing by different device 3 as a consequence of shutting down own device 3b without considering the different device. While this embodiment describes the example of notifying the operator by displaying shutdown confirmation screen 70, information processing device 3b may notify the operator by using voice and sound, for instance.

Shutdown confirmation screen 70 displays first switch 71, second switch 72, and third switch 73, which respectively indicate “YES,” “NO,” and “LINK,” and are capable of accepting input operations by the operator. Information processing device 3b of second apparatus 2b can accept a result of selection through switches 71 to 73 as to whether or not the operator permits the shutdown of information processing device 3b of second apparatus 2b. In addition, information processing device 3b of second apparatus 2b can also accept a result of selection as to whether or not different device 3 selecting the module stored in own device 3b is to be shut down together with own device 3b.

In step S503, when information processing device 3b of second apparatus 2b accepts the input operation using first switch 71 indicating YES, information processing device 3b proceeds to step S506 and executes the shutdown of own device 3b (step S506).

In step S503, when information processing device 3b of second apparatus 2b accepts the input operation using second switch 71 indicating NO, information processing device 3b proceeds to step S505 to stop the shutdown of own device 3b (step S505), and terminates the processing.

Meanwhile, in step S503, when information processing device 3b of second apparatus 2b accepts the input operation using third switch 73 indicating LINK, information processing device 3b proceeds to step S507 and provides a shutdown instruction to different device 3 selecting the module stored in own device 3b (step S507). Information processing device 3a of first apparatus 2a provided with the shutdown instruction executes shutdown of own device 3a (step S508). Information processing device 3b of second apparatus 2b proceeds to step S506 and executes the shutdown of own device 3b (step S506).

As described above, information processing device 3b of second apparatus 2b can shut down not only own device 3b but also different device 3 together, which selects the module in own device 3b.

The above embodiment describes the example of the case in which information processing device 3b of second apparatus 2b determines in step S502 that information processing device 3a of first apparatus 2a selects the module in information processing device 3b. In the meantime, even when information processing device 3b of second apparatus 2b selects the module in first apparatus 2a, the processing proceeds from step S502 to step S503 in which the shutdown confirmation screen is displayed on display unit 12. Subsequently, the processing similar to the above-described processing is performed.

In the latter case, assuming that the first apparatus 2a is activated solely for the purpose of executing the processing of information processing device 3b of second apparatus 2b, it is possible to shut down first apparatus 2a by providing the shutdown instruction from second apparatus 2b side without affecting other processing when the processing of information processing device 3b is completed. Accordingly, the operator of second apparatus 2b can easily shut down first apparatus 2a, which is activated solely for the purpose of executing the processing of second apparatus 2b, without directly operating first apparatus 2a.

### 4. Effects

According to analytical apparatus system 1 of this embodiment, information processing device 3 of each specimen analytical apparatus 2 can process an event that occurs in own device 3 by using not only the modules in own device 3 but also the modules in different devices 3. For this reason, a module having compatibility with other devices does not always have to be installed on all information processing devices 3. Instead, by installing the module on one of information processing devices 3, all information processing devices 3 can share the module. As a consequence, it is possible to reduce waste of resources caused by installing the modules having the same function on all devices 3 in the system in an overlapping manner.

Meanwhile, modules having the same function may cause functional restrictions because their versions are different. Even in this case, certain device 3 can use the module of the latest version without installing the module of the latest version on own device 3 but instead by using the module in different device 3 on which the module of the latest version is installed. Thus, it is possible to reduce functional restrictions and the like attributed to the difference in version.

### 5. Different Embodiment

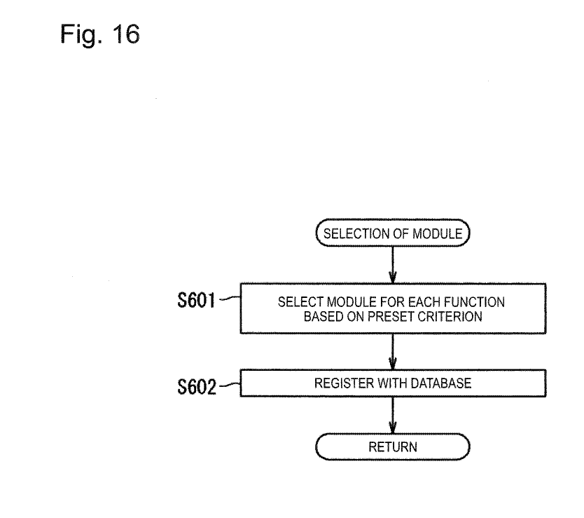

FIG. 16 is a flowchart illustrating procedures of module selection processing in a modified example. In the above-described embodiment, information processing device 3 of specimen analytical apparatus 2 causes display unit 12 to display function selection screen 40, and accepts the selection of the modules by the operator via function selection screen 40. On the other hand, in the modified example of FIG. 16, an information processing device stores a selection criterion for selecting a module in advance, and is configured to automatically select the module on the basis of the selection criterion.

The flowchart illustrated in FIG. 16 represents a modified example of step S113 in FIG. 5. Information processing device 3 of specimen analytical apparatus 2 automatically selects the module for each function out of the modules stored in own device 3 and the modules stored in different devices 3 extracted in step S112 (FIG. 5), on the basis of a preset criterion (step S601).

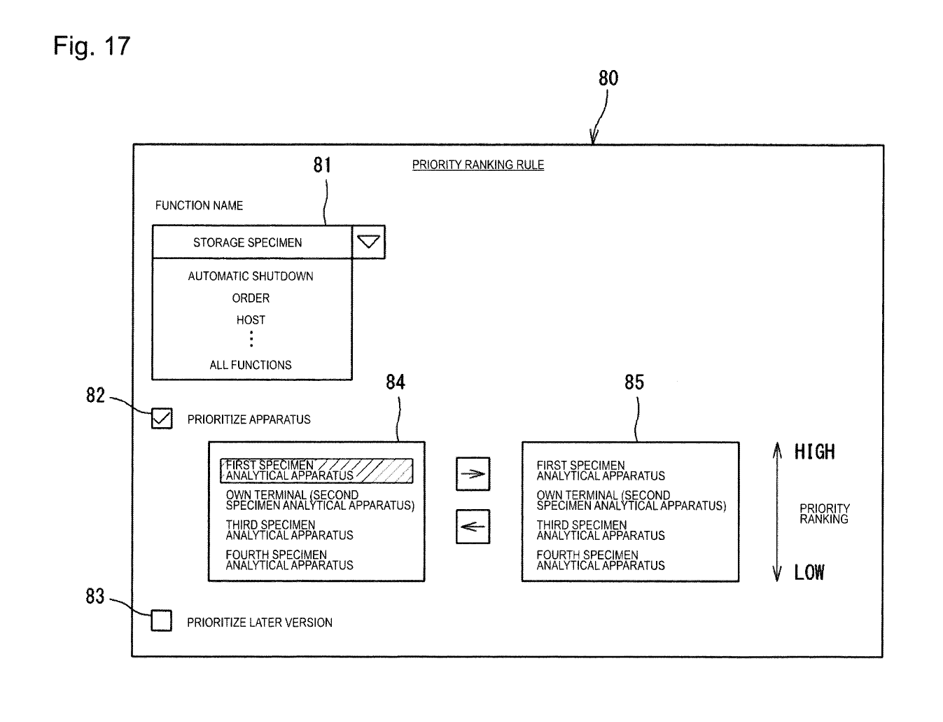

The selection criterion is set by an input operation of the operator. FIG. 17 is a view illustrating an example of an input screen for accepting setting of a selection criterion for a module.

Input screen 80 is provided with drop-down menu 81 for selecting a function, and checkboxes 82 and 83 for selecting whether a priority ranking provided to the apparatus is to take priority or a module with a later version is to take priority. In addition, input screen 80 is provided with option box 84 in which the names of the apparatuses to be provided with the priority ranking are listed up as options, and setting box 85 designed to arrange the names of the apparatuses selected from those in option box 84 and to set the priority ranking among the apparatuses on the basis of the order of arrangement.

Drop-down menu 81 displays a list of functions of the module. Drop-down menu 81 can accept a selection result by the operator who selects one of the functions. The input display below drop-down menu 81 is configured to accept setting concerning the module having the function selected from drop-down menu 81. Accordingly, when “storage specimen” is selected from drop-down menu 81, for example, all other display sections, namely, checkboxes 82 and 83, option box 84, and setting box 85 accept settings concerning the storage specimen module that corresponds to “storage specimen.” In this way, the priority ranking of the module set for each function by the operator can be accepted.

Checkboxes 82 and 83 are the display sections for accepting the selection between giving priority to the priority ranking of each apparatus and giving priority to the later version. One of checkboxes 82 and 83 is subjected to check input. When the input is performed by checking “prioritize apparatus,” information processing device 3 accepts the selection of giving priority to the priority ranking set to each apparatus. When the input is performed by checking “prioritize later version,” information processing device 3 accepts the selection of giving priority to the later version over the priority ranking of each apparatus.

Option box 84 displays the names of the apparatuses to be arranged in setting box 85. The names of the apparatuses can be arranged in setting box 85 in an arbitrary order. Information processing device 3 can accept the order of the names of the apparatus arranged in setting box 85 as the priority ranking of the apparatuses.

As described above, information processing device 3 can accept the contents of the selection criterion set by the operator as the ranking of the module for each function.

Back to FIG. 16, when information processing device 3 selects the module on the basis of the selection criterion in step S601, information processing device 3 registers a selection result with the module database (step S602), and terminates the processing.

According to this modified example, information processing device 3 can automatically select one of the modules having the same function on the basis of the preset selection criterion.

When the operator selects the module by manual input as described in the embodiment, the operator has to reselect the module every time a new module is installed on any of the different devices. In contrast, according to the modified example, the module can be automatically selected in accordance with the preset selection criterion. For this reason, the operator does not have to input the selection result, and system operability can be thus improved.

### 6. Other Embodiments

The invention is not limited only to the above-described embodiments.

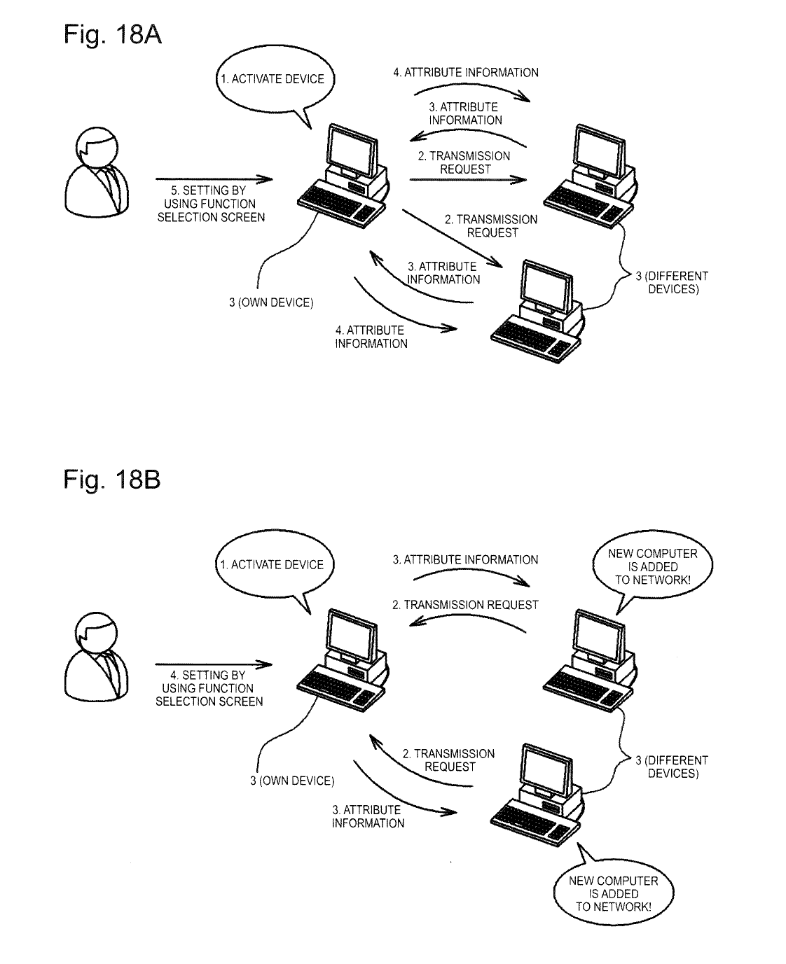

Each of the above embodiments describes the case, as illustrated in FIG. 18A, as an aspect of transmission and reception of the attribute information between information processing devices 3. Here, when information processing device 3 is activated (“1. ACTIVATE DEVICE” in FIG. 18A), activated information processing device 3 transmits the transmission request for the attribute information to different devices 3 other than own device 3 (“2. TRANSMISSION REQUEST” in FIG. 18A). In this case, each different device 3 which receives the transmission request transmits the attribute information on different device 3 in response to the transmission request (“3. ATTRIBUTE INFORMATION” in FIG. 18A). On the other hand, own device 3 which receives the attribute information from different device 3 transmits the attribute information on own device 3 to different device 3 (“4. ATTRIBUTE INFORMATION” in FIG. 18A). Thus, it is possible to exchange the attribute information between devices 3.

In the meantime, as illustrated in FIG. 18B, for example, different device 3 activated beforehand and connected to a network may be configured to constantly monitor connection of new device 3 onto the network. Meanwhile, when own device 3 is activated (“1. ACTIVATE DEVICE” in FIG. 18B), different device 3 may be configured to detect presence of own device 3 on the network, and to transmit the attribute information on different device 3 to detected own device 3 (“2. ATTRIBUTE INFORMATION” in FIG. 18B). In this case again, own device 3 which receives the attribute information from different device 3 transmits the attribute information on own device 3 to different device 3 (“3. ATTRIBUTE INFORMATION” in FIG. 18B). Thus, it is possible to exchange the attribute information between devices 3.

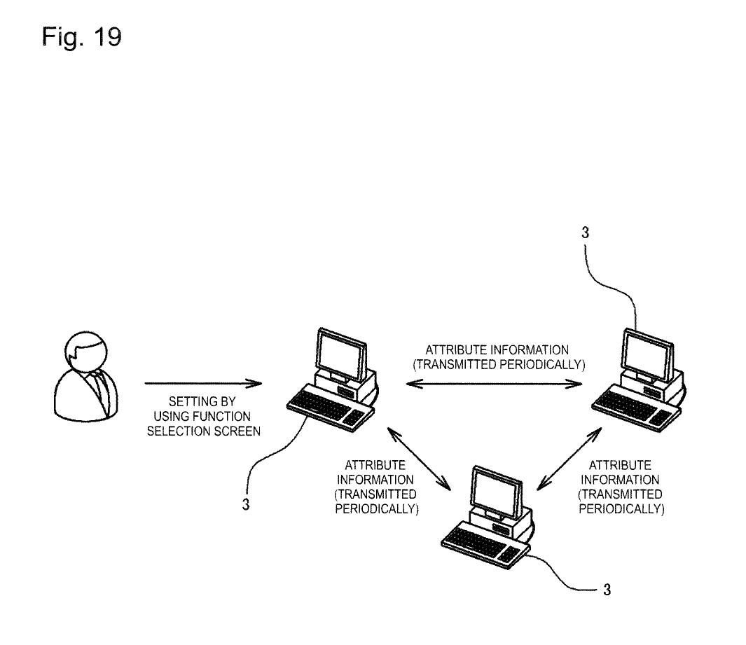

Furthermore, as illustrated in FIG. 19, each device 3 may be configured to transmit the attribute information periodically while device 3 is activated. In this case, each device 3 performs transmission and reception of the attribute information as needed. Accordingly, when new device 3 is connected to the network, new device 3 starts periodical transmission of the attribute information and different device 3 also transmit the attribute information as needed. Thus, it is possible to exchange the attribute information between devices 3 without transmitting the transmission request, monitoring connection of new device 3, or the like.

Meanwhile, each of the above embodiments describes information processing device 3 of specimen analytical apparatus 2. Here, for instance, stand-alone information processing device 3e not attached to specimen analytical apparatus 2 may also have a configuration similar to information processing device 3 of specimen analytical apparatus 2. Accordingly, information processing device 3e can not only use modules in own device 3e but also selectively use modules in different devices 3 other than own device 3e within network N.

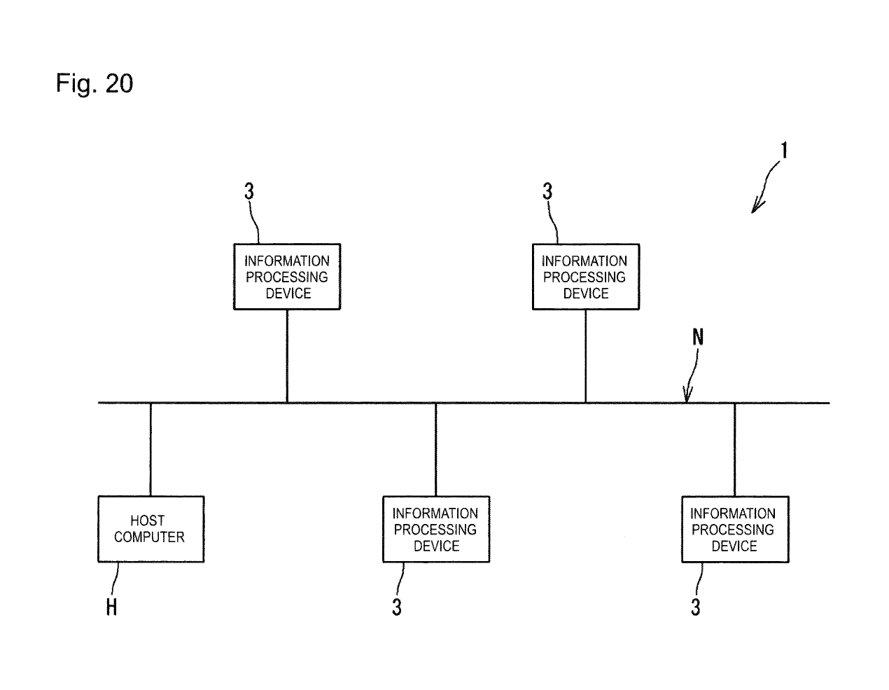

Furthermore, in this system, every information processing device 3 connected to network N may be stand-alone information processing device 3 not attached to specimen analytical apparatus 2 as illustrated in FIG. 20. In this case again, each information processing device 3 can not only use modules in own device 3e but also selectively use modules in different devices 3 other than own device 3e within network N. Thus, the modules installed in respective information processing devices 3 can be appropriately used in the entire system.

The invention includes other embodiments in addition to the above-described embodiments without departing from the spirit of the invention. The embodiments are to be considered in all respects as illustrative, and not restrictive. The scope of the invention is indicated by the appended claims rather than by the foregoing description. Hence, all configurations including the meaning and range within equivalent arrangements of the claims are intended to be embraced in the invention.

1. An analytical apparatus system comprising:
a first specimen analytical apparatus including
a first measurement unit that measures a specimen, and
a first information processing unit that processes an information regarding measurement of the specimen; and
a second specimen analytical apparatus communicably connected to the first specimen analytical apparatus, the second specimen analytical apparatus including
a second measurement unit that measures a specimen, and
a second information processing unit that processes an information regarding of measurement the specimen, wherein
each of the first information processing unit and the second information processing unit includes
a storage unit that stores programs to execute the information processing, and attribute information indicating attributes of the programs, and
a processing unit that executes the programs stored in the storage unit and processes an information regarding to measuring the specimen, and
the processing unit of the first specimen analytical apparatus acquires the attribute information on the programs stored in the second specimen analytical apparatus,
extracts programs, which are applicable to processing of an event occurring in the first specimen analytical apparatus, out of the programs stored in the second specimen analytical apparatus on the basis of the attribute information acquired from the second specimen analytical apparatus, and
when the event occurs in the first specimen analytical apparatus, transmits a request to process the event to the second specimen analytical apparatus, which stores a program selected from the extracted programs.

2. The analytical apparatus system according to claim 1, wherein the processing unit of the second specimen analytical apparatus, which receives the request to process the event, processes the event by executing the selected program.

3. The analytical apparatus system according to claim 1, wherein
the attribute information at least includes function types of the programs and information on versions of the programs, and
the second specimen analytical apparatus extracts the programs applicable to the processing of the event occurring in the first specimen analytical apparatus, on the basis of the function types and the information on the versions.

4. The analytical apparatus system according to claim 1, wherein the processing unit sends the second specimen analytical apparatus a request to transmit the attribute information.

5. The analytical apparatus system according to claim 1, wherein the processing unit accepts a selection of a program by an operator out of the programs stored in the first specimen analytical apparatus and the programs in the second specimen analytical apparatus extracted as applicable, and processes the event by using the selected program.

6. The analytical apparatus system according to claim 5, wherein
the processing unit is capable of displaying a function selection screen designed to accept the selection of the program by the operator,
the function selection screen includes regions respectively corresponding to the information processing units connected to the system, and
each of the regions displays names of the programs stored in the corresponding apparatus in such a way that the programs are selectable.

7. The analytical apparatus system according to claim 1, wherein, for each function, the processing unit automatically selects one of the programs provided with the same function on the basis of a preset selection criterion.

8. The analytical apparatus system according to claim 1, wherein, when the processing unit accepts a shutdown instruction of the first specimen analytical apparatus and any of the programs stored in the first specimen analytical apparatus is selected by the second specimen analytical apparatus, the processing unit outputs to an operator a notice indicating that the program stored in the first specimen analytical apparatus is selected by the second specimen analytical apparatus.

9. The analytical apparatus system according to claim 8, wherein
the processing unit is capable of accepting a selection by the operator as to whether or not to permit the shutdown after the output of the notice, and
the processing unit executes the shutdown when the processing unit accepts a first selection permitting the shutdown, and stops the shutdown when the processing unit accepts a second selection not permitting the shutdown.

10. The analytical apparatus system according to claim 9, wherein
the processing unit is further capable of accepting a third selection by the operator to shut down the second specimen analytical apparatus together with the first specimen analytical apparatus after the output of the notice, and
when the processing unit accepts the third selection, the processing unit transmits a shutdown instruction to the second specimen analytical apparatus, which selects the program stored in the first specimen analytical apparatus.

11. An analytical apparatus system in which specimen analytical apparatuses are communicably connected to each other, wherein
each specimen analytical apparatus comprises:
a measurement unit that measures a specimen; and
an information processing unit that processes an information regarding of measurement the specimen,
each information processing unit includes
a storage unit that stores programs to execute the information processing, and attribute information indicating attributes of the programs, and
a processing unit that executes the programs stored in the storage unit,
the processing unit of one of the apparatuses broadcasts a request to transmit attribute information to different apparatuses connected to the system,
the processing units of the different apparatuses, which receive the transmission request, transmit the attribute information on the programs stored in the different apparatuses to the processing unit of the one apparatus, and
the processing unit in the one apparatus extracts programs, which are applicable to processing of an event occurring in the one apparatus, on the basis of the attribute information acquired from the different apparatuses, and transmits a request to process the event occurring in the one apparatus to any of the different apparatuses storing at least one program selected from the extracted programs.

12. The analytical apparatus system according to claim 11, wherein the processing unit of the different apparatus, which receives the request to process the event, processes the event by executing the selected program.

13. The analytical apparatus system according to claim 11, wherein
the attribute information at least includes function types of the programs and information on versions of the programs, and
the processing unit extracts the programs applicable to the processing of the event occurring in the one apparatus, on the basis of the function types and the information on the versions.

14. The analytical apparatus system according to claim 11, wherein, when the processing unit accepts a shutdown instruction of the one apparatus and any of the programs stored in the one apparatus is selected by the different apparatuses, the processing unit outputs to an operator a notice indicating that the program stored in the one apparatus is selected by the different apparatuses.

15. The analytical apparatus system according to claim 11, wherein, for each function, the processing unit automatically selects one of the programs provided with the same function on the basis of a preset selection criterion.

16. A method of processing an event concerning information processing occurring in a system in which specimen analytical apparatuses are communicably connected to each other, the specimen analytical apparatuses each including an information processing unit configured to perform the information processing concerning a specimen measurement, the method executed by one of the information processing units, the method comprising:
acquiring attribute information on programs stored in a different information processing unit in the system;
extracting programs, which are applicable to processing of an event occurring in the apparatus including the one information processing unit, on the basis of the attribute information acquired from the different information processing unit; and
when the event occurs in the apparatus including the one information processing unit, transmitting a request to process the occurring event to the different apparatus storing a program selected from programs applicable to the apparatus including the one information processing unit.

17. The method of processing an event concerning information processing occurring in a system in which analytical apparatuses are communicably connected to each other according to claim 16, wherein the event is processed by executing the selected program.

18. The method of processing an event concerning information processing occurring in a system in which analytical apparatuses are communicably connected to each other according to claim 16, wherein
the attribute information at least includes function types of the programs and information on versions of the programs, and
the programs applicable to the processing of the event occurring in the apparatus including the one information processing unit are extracted on the basis of the function types and the information on the versions.

19. The method of processing an event concerning information processing occurring in a system in which analytical apparatuses are communicably connected to each other according to claim 16, wherein the processing unit is configured to send the different apparatus a request to transmit the attribute information.

20. The method of processing an event concerning information processing occurring in a system in which analytical apparatuses are communicably connected to each other according to claim 16, wherein, for each function, the processing unit automatically selects one of the programs provided with the same function on the basis of a preset selection criterion.
